(19) United States
(12) Patent Application Publication
(10) Pub. No.: US 2007/0053892 A1
(43) Pub. Date: Mar. 8, 2007

(54) Methods and kits for co-administration of nutritional supplements

(75) Inventors: John A. Giordano, West Orange, NJ (US); Charles J. Balzer, Lavalette, NJ (US)

Correspondence Address: Kirkpatrick & Lockhart Preston Gates Ellis LLP, 1735 NEW YORK AVENUE, NW, SUITE 500, WASHINGTON, DC, 20006, US

(73) Assignee: Everett Laboratories, Inc.

(21) Appl. No.: 11/219,745

(22) Filed: Sep. 7, 2005

(51) Int. Cl. A61K 38/43; A61K 31/714; A61K 31/60; A61K 31/525; A61K 31/4415; A61K 31/122

(52) U.S. Cl. 424/94.1; 514/165; 514/251; 514/52; 514/350; 514/690

(57) ABSTRACT

The present invention relates to methods of co-administration of various vitamin, mineral and aspirin compositions, and in a specific embodiment, comprise co-administering one composition comprising vitamin B₆, vitamin B₁₂, folic acid, and CoQ₁₀ and a second composition comprising aspirin for nutritional supplementation and drug therapy in order to prevent, treat and/or alleviate the occurrence or negative effects of cardiovascular disease; and kits provided for co-administration of various vitamin, mineral and drug compositions, and in a specific embodiment, comprise one composition comprising vitamin B₆, vitamin B₁₂, folic acid, and CoQ₁₀ and a second composition comprising aspirin for nutritional supplementation and drug therapy in order to prevent, treat and/or alleviate the occurrence or negative effects of cardiovascular disease.

### FIELD OF THE INVENTION

The present invention relates to methods of co-administration of various vitamin, mineral and aspirin compositions and kits provided for co-administration of these compositions for nutritional supplementation and therapy in order to prevent, treat and/or alleviate the occurrence or negative effects of cardiovascular disease including congestive heart failure, ischemic heart disease, cardiomyopathy, hypertension, coronary artery disease, high blood pressure, arrhythmia, thrombogenicity, angina, myocardial infarction, and stroke.

### BACKGROUND OF THE INVENTION

Cardiovascular disease continues to be the number one cause of death for both men and women in the United States. Statistics Related to Heart Disease, available at www.health.uab.edu/show.asp?durki=39661 (last visited 24 Aug. 2005). Correlated risk factors for the incidence of cardiovascular disease include blood level elevations of both cholesterol and homocysteine. Id. Recent developments in nutritional research suggest that nutritional supplementation with specific vitamins and minerals, as an adjunct to proper diet, exercise and medical care, can aid in preventing, treating and/or alleviating the occurrence or negative effects of this disease. Further, research suggests that long-term low-dose administration of aspirin (acetyl salicylic acid) can aid in preventing, treating and/or alleviating the occurrence or negative effects of cardiovascular disease as well.

Nutrition plays a critical role in maintaining good health, and nutritional supplementation serves a vital role in protecting against poor nutrition and disease. For example, recent research has shown that vitamins and minerals help to prevent, treat and/or alleviate the occurrence or negative effects of cardiovascular disease. While supplementation with certain vitamins and minerals protects against the onset of these diseases, other vitamins and minerals have been found to inhibit the beneficial effects of these certain vitamins and minerals. Specifically, vitamin B6, vitamin B12, folic acid and coenzyme Q10 (“CoQ10”) play integral roles in physiological mechanisms that serve to prevent, treat and/or alleviate the occurrence or negative effects of cardiovascular disease. Supplementation with other vitamins and minerals may inhibit the beneficial effects of vitamin B6, vitamin B12, folic acid and CoQ10. Thus, when choosing or administering a nutritional supplement, it is essential to understand the physiological needs and risks of individual patients and population groups and the interactions between various vitamins and minerals.

Aspirin is now accepted as an important weapon in the prevention of heart disease. For example, recent research has shown that aspirin therapy helps to prevent, treat and/or alleviate the occurrence or negative effects of cardiovascular disease, available at http://www.mayoclinic.com/invoke.cfm?id=HQ00269 (last visited 24 Aug. 2005).

Thus, administration of aspirin in conjunction with nutritional supplementation can benefit patients suffering from cardiovascular disease.

### SUMMARY OF THE INVENTION

The present invention provides methods of co-administering prophylactic and therapeutic compositions and kits comprising therapeutic and prophylactic compositions. Specifically, the present invention includes vitamins and minerals that prevent, treat and/or alleviate the occurrence or negative effects of cardiovascular disease, especially congestive heart failure, ischemic heart disease, cardiomyopathy, hypertension, coronary artery disease, high blood pressure, arrhythmia, thrombogenicity, angina, myocardial infarction, and stroke. The present invention also may be formulated to exclude vitamins and minerals known or suspected to inhibit the beneficial effects of the included vitamins and minerals. The present invention further specifically includes aspirin that prevents, treats and/or alleviates the occurrence or negative effects of cardiovascular disease.

The present invention includes methods of co-administering the compositions of the invention to patients, in any order, to prevent, treat and/or alleviate the occurrence or negative effects of cardiovascular disease including congestive heart failure, ischemic heart disease, cardiomyopathy, hypertension, coronary artery disease, high blood pressure, arrhythmia, thrombogenicity, angina, myocardial infarction, and stroke.

In one embodiment of the present invention, the methods may utilize compositions in a swallowable form. In another embodiment, the methods may utilize compositions substantially free of one or more of other added vitamins, added minerals, and added coenzymes. In another embodiment, the methods may utilize compositions comprising pharmaceutically acceptable carriers, such as one or more of binders, diluents, lubricants, glidants, colorants, emulsifiers, disintegrants, starches, water, oils, alcohols, preservatives and sugars.

In one embodiment of the present invention the methods may utilize compositions comprising vitamin B6, vitamin B12, folic acid, and CoQ10 and compositions comprising aspirin. In another embodiment, methods of the present invention may include folic acid in the form of vitamin B9, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In another embodiment, the methods may utilize compositions comprising about 15 mg to about 45 mg vitamin B6; about 0.9 mg to about 2.7 mg vitamin B12; about 1.25 mg to about 3.75 mg folic acid; and about 25 mg to about 75 mg CoQ10.

In yet another embodiment, the methods may utilize compositions comprising about 24 mg to about 36 mg vitamin B6; about 1.44 mg to about 2.16 mg vitamin B12; about 2 mg to about 3 mg folic acid; and about 40 mg to about 60 mg CoQ10.

In another embodiment, the methods may utilize compositions comprising about 27 mg to about 33 mg vitamin B6; about 1.26 mg to about 1.98 mg vitamin B12; about 2.25 mg to about 2.75 mg folic acid; and about 45 mg to about 55 mg CoQ10.

In another embodiment, the methods may utilize compositions comprising about 30 mg vitamin B6; about 1.8 mg vitamin B12; about 2.5 mg folic acid; and about 50 mg CoQ10. In one embodiment, the methods may utilize compositions comprising about 40.5 mg to about 121.5 mg aspirin. In another embodiment, the methods may utilize compositions comprising about 64.8 mg to about 97.2 mg aspirin. In yet another embodiment, the methods may utilize compositions comprising about 72.9 mg to about 89.1 mg aspirin. Alternatively, the methods may utilize compositions comprising about 81 mg aspirin.

In one embodiment, the methods may comprise co-administering to a patient one unit dose of compositions comprising vitamin B6, vitamin B12, folic acid, and CoQ10 and one unit dose of compositions comprising aspirin, at the same time.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 1 minute following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 2 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 3 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 4 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 5 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 6 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 7 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 8 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 9 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 10 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 11 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 12 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 13 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 14 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 15 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 16 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 17 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 18 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 19 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 20 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 21 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 22 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 23 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 24 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 25 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 26 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 27 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 28 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 29 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 30 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 31 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 32 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 33 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 34 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 35 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 36 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 37 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 38 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 39 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 40 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 41 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 42 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 43 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 44 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 45 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 46 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 47 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 48 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 49 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 50 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 51 minutes following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 52 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 53 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 54 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 55 minutes following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 56 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 57 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 58 minutes following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 59 minutes following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 60 minutes following the administration of the other.

In one embodiment, the methods may comprise co-administering to a patient one unit dose of compositions comprising vitamin B6, vitamin B12, folic acid, and CoQ10 and one unit dose of compositions comprising aspirin, in either order, one administered about 2 hours following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 3 hours following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 4 hours following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 5 hours following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 6 hours following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 7 hours following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 8 hours following the administration of the other.

In an alternative embodiment, the methods may comprise co-administering to a patient one unit dose of compositions comprising vitamin B6, vitamin B12, folic acid, and CoQ10 and one unit dose of compositions comprising aspirin, in either order, one administered about 9 hours following the administration of the other.

In another embodiment, the methods may comprise co-administering to a patient one unit dose of compositions comprising vitamin B6, vitamin B12, folic acid, and CoQ10 and one unit dose of compositions comprising aspirin, in either order, one administered about 10 hours following the administration of the other.

In yet another embodiment, the methods may comprise co-administering to a patient one unit dose of compositions comprising vitamin B6, vitamin B12, folic acid, and CoQ10 and one unit dose of compositions comprising aspirin, in either order, one administered about 11 hours following the administration of the other.

In another specific embodiment, the methods may comprise co-administering to a patient one unit dose of compositions comprising vitamin B6, vitamin B12, folic acid, and CoQ10 and one unit dose of compositions comprising aspirin, in either order, one administered about 12 hours following the administration of the other.

In one embodiment, the methods may comprise co-administering to a patient one unit dose of compositions comprising vitamin B6, vitamin B12, folic acid, and CoQ10 and one unit dose of compositions comprising aspirin, in either order, together once a day.

In one embodiment, the methods may comprise co-administering to a patient one unit dose of compositions comprising vitamin B6, vitamin B12, folic acid, and CoQ10 and one unit dose of compositions comprising aspirin, in either order, together twice a day.

In one embodiment, the methods may comprise co-administering to a patient one unit dose of compositions comprising vitamin B6, vitamin B12, folic acid, and CoQ10 and one unit dose of compositions comprising aspirin, in either order, together three times a day.

In one embodiment, the methods may comprise co-administering to a patient one unit dose of compositions comprising vitamin B6, vitamin B12, folic acid, and CoQ10 and one unit dose of compositions comprising aspirin, in either order, together four times a day.

In an alternative embodiment, the methods may comprise co-administering to a patient one unit dose of compositions comprising vitamin B6, vitamin B12, folic acid, and CoQ10 and one unit dose of compositions comprising aspirin, in either order, together once every 2 days.

In another embodiment, the methods may comprise co-administering to a patient one unit dose of compositions comprising vitamin B6, vitamin B12, folic acid, and CoQ10 and one unit dose of compositions comprising aspirin, in either order, together once every 3 days.

In yet another embodiment, the methods may comprise co-administering to a patient one unit dose of compositions comprising vitamin B6, vitamin B12, folic acid, and CoQ10 and one unit dose of compositions comprising aspirin, in either order, together once every 4 days.

In another embodiment, the methods may comprise co-administering to a patient one unit dose of compositions comprising vitamin B6, vitamin B12, folic acid, and CoQ10 and one unit dose of compositions comprising aspirin, in either order, together once every 5 days.

In another specific embodiment, the methods may comprise co-administering to a patient one unit dose of compositions comprising vitamin B6, vitamin B12, folic acid, and CoQ10 and one unit dose of compositions comprising aspirin, in either order, together once every 6 days.

In another embodiment, the methods may comprise co-administering to a patient one unit dose of compositions comprising vitamin B6, vitamin B12, folic acid, and CoQ10 and one unit dose of compositions comprising aspirin, in either order, together once a week.

The present invention includes kits that may be provided to patients to prevent, treat and/or alleviate the occurrence or negative effects of cardiovascular disease including congestive heart failure, ischemic heart disease, cardiomyopathy, hypertension, coronary artery disease, high blood pressure, arrhythmia, thrombogenicity, angina, myocardial infarction, and stroke.

In one embodiment of the present invention, the kits may comprise compositions in a swallowable form. In another embodiment, the kits may comprise compositions substantially free of one or more of other added vitamins, added minerals, and added coenzymes. In another embodiment, the kits may comprise compositions comprising pharmaceutically acceptable carriers, such as one or more of binders, diluents, lubricants, glidants, colorants, emulsifiers, disintegrants, starches, water, oils, alcohols, preservatives and sugars.

In one embodiment of the present invention, the kits may comprise compositions comprising vitamin B6, vitamin B12, folic acid, and CoQ10 and compositions comprising aspirin packaged for co-administration to a patient, in any order. In another embodiment, the kits may include folic acid in the form of vitamin B9, folacin, metafolin, folate or natural isomers thereof including (6S)-tetrahydrofolic acid, 5-methyl-(6S)-tetrahydrofolic acid, 5-formyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid or polyglutamyl derivatives thereof.

In one embodiment, the kits may comprise compositions comprising about 15 mg to about 45 mg vitamin B6; about 0.9 mg to about 2.7 mg vitamin B12; about 1.25 mg to about 3.75 mg folic acid; and about 25 mg to about 75 mg CoQ10.

In an alternative embodiment, the kits may comprise compositions comprising about 24 mg to about 36 mg vitamin B6; about 1.44 mg to about 2.16 mg vitamin B12; about 2 mg to about 3 mg folic acid; and about 40 mg to about 60 mg CoQ10.

In another embodiment, the kits may comprise compositions comprising about 27 mg to about 33 mg vitamin B6; about 1.26 mg to about 1.98 mg vitamin B112; about 2.25 mg to about 2.75 mg folic acid; and about 45 mg to about 55 mg CoQ10.

In yet another embodiment, the kits may comprise compositions comprising about 30 mg vitamin B6; about 1.8 mg vitamin B12; about 2.5 mg folic acid; and about 50 mg CoQ10.

In one embodiment, the kits may comprise compositions comprising about 40.5 mg to about 121.5 mg aspirin.

In an alternative embodiment, the kits may comprise compositions comprising about 64.8 mg to about 97.2 mg aspirin.

In another embodiment, the kits may comprise compositions comprising about 72.9 mg to about 89.1 mg aspirin.

In yet another embodiment, the kits may comprise compositions comprising about 81 mg aspirin.

In another embodiment, the kits may be packaged in various forms including bottles and blister packs.

In yet another embodiment, the kits may be packaged in bottles that are sold together; one bottle containing compositions comprising vitamin B6, vitamin B12, folic acid, and CoQ10 and one bottle containing compositions comprising aspirin.

In another embodiment, the kits may be packaged in bottles that are sold separately; one bottle containing compositions comprising vitamin B6, vitamin B12, folic acid, and CoQ10 and one bottle containing compositions comprising aspirin.

In an alternative embodiment, the kits may be packaged in bottles advertised as more effective if co-administered; one bottle containing compositions comprising vitamin B6, vitamin B12, folic acid, and CoQ10 and one bottle containing compositions comprising aspirin. The advertisements may consist of internet, print, and product packaging advertisements.

In another embodiment, the kits may be packaged in blister packs that are sold together; one blister pack containing compositions comprising vitamin B6, vitamin B12, folic acid, and CoQ10 and one blister pack containing compositions comprising aspirin.

In yet another embodiment, the kits may be packaged in one blister pack containing compositions comprising vitamin B6, vitamin B12, folic acid, and CoQ10 and containing compositions comprising aspirin paired together per unit dose.

In another embodiment, the kits may be packaged in blister packs that are sold separately; one blister pack containing compositions comprising vitamin B6, vitamin B12, folic acid, and CoQ10 and one blister pack containing compositions comprising aspirin.

In yet another embodiment, the kits may be packaged in blister packs advertised as more effective if co-administered; one blister pack containing compositions comprising vitamin B6, vitamin B12, folic acid, and CoQ10 and one blister pack containing compositions comprising aspirin. The advertisements may consist of internet, print, and product packaging advertisements.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, and excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “swallowable form” refers to any compositions that do not readily dissolve when placed in the mouth and may be swallowed whole without any chewing or discomfort. In one embodiment, may have a shape containing no sharp edges and a smooth, uniform and substantially bubble free outer coating.

The phrase “co-administration” refers to administration of two compositions to a patient together and in a specific embodiment, within a certain desired time.

The phrase, “no added vitamins, no added minerals and no added coenzymes” does not include trace amounts of vitamins, minerals, or coenzymes added during the manufacturing, tableting, or mixing process. These trace amounts of vitamins, minerals or coenzymes are not therapeutically significant.

As stated earlier, cardiovascular disease is the number one cause of death for adults in the United States. Recent developments in nutritional research suggest that nutritional supplementation with certain vitamins and minerals, as an adjunct to proper diet, exercise and medical care, can aid in preventing, treating and/or alleviating the occurrence or negative effects of these diseases. Further, research suggests that administration of aspirin can aid in preventing, treating and/or alleviating the occurrence or negative effects of cardiovascular disease.

The methods and kits of the present invention provide means to optimize good health by utilizing vitamin and mineral combinations and drugs that specifically aim to prevent, treat and/or alleviate the occurrence or negative effects of cardiovascular disease. The methods and kits of the present invention may be administered to or directed to a subject such as a human or any other organism. Each of the added vitamins, minerals and drugs that can be included in the present invention, including vitamin B6, vitamin B12, folic acid, CoQ10, and aspirin, plays a specific role in preventing, treating and/or alleviating the occurrence or negative effects of cardiovascular disease. In a specific embodiment, vitamins, minerals, and coenzymes that inhibit these compounds' beneficial effects, may be specifically excluded from the methods and kits of the present invention. Further, in another specific embodiment, other added vitamins and/or minerals and/or coenzymes can be excluded.

The methods and kits of the present invention comprise compositions that may be co-administered. Co-administration of the compositions of the present invention and/or patient compliance may be found to achieve an improved clinical outcome preventing, treating and/or alleviating the occurrence or negative effects of cardiovascular disease.

Vitamin B6 and vitamin B12 are water-soluble nutrients that generally are not stored in the body. These vitamins play a variety of roles within the body. They are included in the methods and kits of the present invention due to their roles in preventing, treating and/or alleviating the occurrence or negative effects of cardiovascular disease.

Vitamin B6, vitamin B12, and folic acid help prevent, treat and/or alleviate the occurrence or negative effects of cardiovascular disease through their involvement in the metabolism and breakdown of homocysteine. Homocysteine is an intermediary product produced by metabolism of the amino acid methionine. Elevated levels of homocysteine have been correlated to an increased risk of cardiovascular disease. Maxwell, Suppl 1 Basic Res. Cardiol. 165-71 (2003). Elevated levels of homocysteine may lead to increased risk of cardiovascular disease due to this metabolite's numerous deleterious effects on the vascular system including impairing endothelial function, inducing thrombosis, and increasing oxidant stress. Schlaich, 153(2) Atheroscler. 383-89 (2000); Hanratty, 85(3) Heart 326-30 (2001). Efficient breakdown of this intermediary product is necessary to avoid these deleterious effects on the vascular system, and the metabolic breakdown pathways of homocysteine require vitamins B6, B12 and folic acid. Thus, optimizing the levels of these vitamins has cardio-protective effects by promoting the efficient degradation of homocysteine. Haynes, 16(5) Cardiovasc. Drugs Ther. 391-9 (2002).

Folic acid is essential for the formation of red and white blood cells within bone marrow and also plays a role in heme formation. National Research Council, Recommended Dietary Allowances 10th ed., 150 (1989). Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine to methionine. The activation of folic acid requires a vitamin B12-dependent transmethylation and vitamin B12 is also necessary for folic acid delivery to tissues. Id.

Folic acid also may have additional physiological effects beyond its role in homocysteine breakdown that protect against cardiovascular disease. Bailey, 133(6) J. Nutr. 1961S-68S (2003); Doshi, 41(11) Clin. Chem. Lab. Med. 1505-12 (2003); Haynes, supra. For example, folic acid improves the levels and functioning of the health promoting, endothelial-derived compound nitric oxide (NO). Das, 19(7-8) Nutr. 686-92 (2003). Folic acid creates this effect by enhancing the activity of the enzyme nitric oxide synthase, stimulating endogenous tetrahydrobiopterin, and inhibiting generation of intracellular superoxide. All of these actions enhance the half-life of NO thus creating cardioprotective effects. Lucock, 71 Mol. Genet. Metab. 121-38 (2000).

In one embodiment, the methods and kits of the present invention may include vitamin B6 in amounts ranging from about 15 mg to about 45 mg. In another embodiment, the methods and kits of the present invention may include vitamin B6 in amounts ranging from about 24 mg to about 36 mg. In yet another embodiment, the methods and kits of the present invention may include vitamin B6 in amounts ranging from about 27 mg to about 33 mg. In a specific embodiment, the methods and kits of the present invention may include vitamin B6 in an amount of about 30 mg.

Vitamin B12 also may be included in the methods and kits of the present invention. In one embodiment of the present invention, vitamin B12 may be included in amounts ranging from about 0.9 mg to about 2.7 mg. In another embodiment, vitamin B12 may be included in amounts ranging from about 1.44 mg to about 2.16 mg. In yet another embodiment, vitamin B12 may be included in amounts ranging from about 1.26 mg to about 1.98 mg. In a specific embodiment of the present invention, vitamin B12 may be included in an amount of about 1.8 mg.

Folic acid also may be included in the methods and kits of the present invention. In one embodiment of the present invention, folic acid may be included in amounts ranging from about 1.25 mg to about 3.75 mg. In another embodiment of the present invention folic acid may be included in amounts ranging from about 2 mg to about 3 mg. In yet another embodiment, folic acid may be included in amounts ranging from about 2.25 mg to about 2.75 mg. In a specific embodiment of the present invention, folic acid may be included in an amount of about 2.5 mg.

CoQ10 belongs to the ubiquinone family of substances. CoQ10 is a fat soluble, vitamin-like substance that is an essential cofactor in the mitochondrial electron transport chain and thus is vital in the production of Adenosine-Tri-Phosphate (ATP). CoQ10 is biosynthesized in the body and shares a common synthetic pathway with cholesterol. Sarter, 16(4) J Cardiovasc. Nurs. 9-20 (2002).

Early studies have documented a deficiency of CoQ10 in patients with a variety of cardiovascular disorders including, but not limited to, congestive heart failure, ischemic heart disease, cardiomyopathy, and hypertension. Id. Studies have indicated that administration of CoQ10 provides a therapeutic benefit to patients with cardiovascular disease. CoQ10 has been shown to significantly reduce blood pressure in patients with hypertension. Id. Administration of CoQ10 to patients with congestive heart failure has been shown to improve myocardial metabolic function, reduce blood pressure, and reduce myocardial thickness. Id. Further, CoQ10 enhances both the magnitude and rate of clinical improvement in patients with ischemic heart disease. Langsjoen et al., 9(24) Biofactors. 273-284 (1999). Additionally, studies have suggested CoQ10 may be a benefit in treating patients with cardiomyopathy. Patients treated with CoQ10 showed significant improvement in cardiac function including ejection fraction and cardiac output. J Cardiovasc. Nurs. at 9.

Nutritional supplementation with CoQ10 can help prevent, treat and/or alleviate the occurrence or negative effects of cardiovascular disease. In one embodiment, the methods and kits of the present invention may include CoQ10 in amounts ranging from about 25 mg to about 75 mg. In another embodiment, the methods and kits of the present invention may include CoQ10 in amounts ranging from about 40 mg to about 60 mg. In yet another embodiment, the methods and kits of the present invention may include CoQ10 in amounts ranging from about 45 mg to about 55 mg. In a specific embodiment, the methods and kits of the present invention may include CoQ10 in an amount of about 50 mg.

Acetyl salicylic acid, commonly known by its trade name as aspirin, belongs to a family of medications known as nonsteroidal anti-inflammatory drugs (“NSAIDs”). Although aspirin was discovered over a century ago and had been primarily used as a pain, fever, and inflammation reducer, relatively recent research has focused on its protective benefit against cardiovascular disease, available at http://www.mayoclinic.com/invoke.cfm?id=HQ00269 (last visited 24 Aug. 2005).

A key process in the development of cardiovascular disease is plaque buildup along the inner walls of the artery, a process generally known as atherosclerosis. Platelets are tiny cell-like fragments in the blood that are essential for blood clotting. In the process of atherosclerosis, these platelets may overreact to arterial damage and cause excessive clotting (platelet aggregation), and a resultant exacerbation of the atherosclerotic process. Harrison's Principles of Internal Medicine 994-995 (12th ed. 1991). Aspirin prevents blood clotting, thus reducing platelet aggregation by inhibiting the enzyme cyclo-onygenase-1 (COX-1) that produces a key clotting factor known as Thromboxane A-2. Secondary to this role, aspirin has been implicated as playing a key role in reducing the risk of occlusive vascular events. Hennekens et al., 96 Circulation 2751-2753 (1997). Research has shown the beneficial effects of aspirin in treating and preventing ischemic heart disease, coronary thrombosis, angina, myocardial infarction, and stroke, available at http://www.americanheart.org/presenter/jhtml?identifier-4456 (last visited 24 Aug. 2005); http://www.aspirin-foundation.com/uses/crdio.html (last visited 24 Aug. 2005).

Low-dose aspirin therapy can help prevent, treat and/or alleviate the occurrence or negative effects of cardiovascular disease. In one embodiment, the methods and kits of the present invention may include aspirin in amounts ranging from about 40.5 mg to about 121.5 mg. In another embodiment, the methods and kits of the present invention may include aspirin in amounts ranging from about 64.8 mg to about 97.2 mg. In yet another embodiment, the methods and kits of the present invention may include aspirin in amounts ranging from about 72.9 mg to about 89.1 mg. In a specific embodiment, the methods and kits of the present invention may include aspirin in an amount of about 81 mg.

High cholesterol is a risk factor for cardiovascular disease. Cholesterol assists in the production of new cells, insulates nerves and produces hormones. Normally, the liver makes the necessary amount of cholesterol for these functions. However, cholesterol enters the body from dietary sources, such as animal-based foods like milk, eggs and meat. Too much cholesterol in the body is a major risk factor for coronary artery disease. When too much cholesterol is in the blood, it builds up in the walls of the arteries. Over time, this build-up causes atherosclerosis. The arteries become narrowed and blood flow to the heart is slowed or blocked. Ultimately, this blockage can result in a heart attack. Two types of cholesterol include low-density lipoprotein (“LPL”) and high-density lipoprotein (“HDL”). LDL is the main source of plaque that clogs the arteries. The main goal in lowering cholesterol is to lower the amount of LDL and raise HDL levels. Cholesterol lowering medications assist in lowering LDL levels and raising HDL levels, available at http://my.webmd.com/content/pages/9/1675_{—}57815 (last visited 25 Aug. 2005).

Cholesterol-lowering drugs include statins, niacin, bile-acid resins and fibric acid derivatives. Statins, the most common form of cholesterol-lowering drugs and often the first line of treatment, block the production of cholesterol in the liver, lower LDL amounts and raise HDL levels. Examples of statins include, Crestor®, Lipitor®, Lescol®, Mevacor® and Zocor®. Id. Co-administration of cholesterol-lowering drugs with aspirin and/or compositions comprising vitamin B6, vitamin B12, folic acid, and CoQ10 can help prevent, treat and/or alleviate the occurrence or negative effects of cardiovascular disease.

The active ingredients are available from numerous commercial sources, and in several active forms or salts thereof, known to those of ordinary skill in the art. Hence, the methods and kits of the present invention are not limited to comprising or using any particular form of the vitamin, mineral or drug ingredient described herein.

Nutrition is a constantly evolving health science. Nearly as proliferative as research findings correlating nutrients and disease prevention are findings demonstrating that supplementation with some nutrients can be counter-productive to the health needs of specific populations. In a specific embodiment, the methods and kits of the present invention may be substantially free of other added vitamins, minerals, and coenzymes.

The compositions of the present invention are preferably administered in amounts to patients that provide the supplementation required to alleviate the vitamin deficiencies associated with cardiovascular disease and optimize the effectiveness of the drug therapy in alleviating symptoms of cardiovascular disease. An exemplary dosage of the compositions of the present invention may consist of one or more caplets for human oral consumption. If more than one caplet is used, each individual caplet may be identical to the other caplets, or each may contain only some of the ingredients of the composition, so that the combination of the different caplets comprises a composition of the present invention.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention, the swallowable compositions may have a shape containing no sharp edges and a smooth, uniform and substantially bubble free outer coating.

A specific embodiment of the present invention may comprise swallowable compositions comprising vitamin B6, vitamin B12, folic acid, and CoQ10 in caplet form. Another specific embodiment of the present invention may comprise swallowable compositions comprising aspirin in caplet form.

To prepare the swallowable compositions in caplet form, each of the active ingredients may be combined in intimate admixture with a suitable carrier according to conventional compounding techniques. In a specific embodiment of swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia. Pharmaceutical carriers and formulations for swallowable compounds are well known to those of ordinary skill in the art. See generally, e.g., Wade & Waller, Handbook of Pharmaceutical Excipients (2nd ed. 1994).

Disintegrants also may be included in the compositions of the present invention in order to facilitate dissolution. Disintegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include by way of example and without limitation, starches such as corn starch, potato starch, pre-gelatinized and modified starches thereof, cellulosic agents such as Ac-di-sol, montmorrilonite clays, cross-linked PVP, sweeteners, bentonite, microcrystalline cellulose, croscarmellose sodium, alginates, sodium starch glycolate, gums such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium, and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol, and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, and polyols of less than 13 carbon atoms, such as mannitol, xylitol, sorbitol, maltitol, and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents such as grape skin extract, beet red powder, beta carotene, annato, carmine, turmeric, paprika, and others known to those of ordinary skill in the art. It is recognized that no colorant is required in the nutritional supplement compositions described herein.

If desired, compositions may be sugar coated or enteric coated by standard techniques.

The swallowable compositions of the present invention may be prepared using conventional methods and materials known in the pharmaceutical art. For example, U.S. Pat. Nos. 5,215,754 and 4,374,082 relate to methods for preparing swallowable compositions. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in Wade & Waller, supra. All active ingredients, fillers and excipients are commercially available from companies such as Aldrich Chemical Co., FMC Corp, Bayer, BASF, Alexi Fres, Witco, Mallinckrodt, Rhodia, ISP, and others.

A specific embodiment of the present invention may comprise swallowable compositions packaged in blister packs. Blister packs as packaging for swallowable compositions are well known to those of ordinary skill in the art. Blister packs may be made of a transparent plastic sheet which as been formed to carry a matrix of depression or blisters. One or more swallowable compositions are received in each depression or blister. A foil or plastic backing is then adhered across the plane of the sheet sealing the swallowable compositions in their respective blisters. Examples of materials used for the blister packs include, but are not limited to, aluminum, paper, polyester, PVC, and polypropylene. Alternative materials are known to those of ordinary skill in the art. To remove a swallowable composition, the depression material is pressed in and the composition is pushed through the backing material. Multiple blister packs may be placed in an outer package, often a box or carton for sale and distribution.

Another specific embodiment of the present invention may comprise swallowable compositions packaged in bottles. The bottle may be glass or plastic in form with a pop or screw top cap. Bottle packaging for compositions in swallowable form are well known to those of ordinary skill in the art.

Additionally, the unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

Kits are commonly used for dispensing pharmaceutical actives and are known in the pharmaceutical art. Kits were developed and designed to administer multiple doses of the same active ingredient or for the concurrent or nonconcurrent administration of two or more active agents. See e.g. U.S. Pat. No. 6,024,222, to Friberg et al., issued Feb. 15, 2000; U.S. Pat. No. 6,219,997, to Friberg et al., issued Apr. 24, 2001; U.S. Pat. Pub. 2003/0168376 A1, Taneja et al. published Sep. 11, 2003; U.S. Pat. Pub. 2003/0111479, Taneja et al., published Jun. 19, 2003; U.S. Pat. No. 6,375,956, to Hermelin et al., issued Apr. 23, 2002; PCT Pub. WO 88/02342, Astra Lakemedel Aktiebolag, published Apr. 7, 1988; U.S. Pat. No. 4,295,567, to Knudsen, issued Oct. 20, 1981; DE 29719 070, to Byk Gulden Lomberg Chemische Fabrik, published Jun. 25, 1998; U.S. Pat. No. 5,848,976, to Weinstein, issued Dec. 15, 1998; U.S. Pat. No. 6,270,796, to Weinstein, issued Aug. 7, 2001; U.S. Pat. No. 6,564,945, to Weinstein et al., issued May 20, 2003; and U.S. Pat. No. 5,788,974, to D'Amico et al., issued Aug. 4, 1998.

A specific embodiment of the present invention may comprise kits wherein the packaging may consist of bottles and blister packs. In another embodiment, the kits may contain bottles that are sold together, one bottle containing a first composition and a second bottle containing a second composition. In a further embodiment, the kits may contain bottles that are sold separately; one bottle containing a first composition and a second bottle containing a second composition. In yet another embodiment, the kits may contain bottles containing a first composition and a second composition that are advertised as more effective if co-administered. In another embodiment, the advertisements of the kits may consist of internet, print, and product packaging advertisements. In a further embodiment, the kits may contain blister packs that are sold together wherein the blister packs may contain a first blister pack containing a first composition and a second blister pack containing a second composition. In another embodiment, the kits may contain blister packs that contain both a first composition and a second composition paired together per unit dose. In yet another embodiment, the kits may contain blister packs that are sold separately that comprise a first blister pack containing a first composition and a second blister pack containing a second composition. In another embodiment, the kits may contain blister packs containing a first composition and a second composition that are advertised as more effective if co-administered. In a further embodiment, the kits may contain a first composition and a second composition that may be co-administered to a patient. In another embodiment, the kits may contain a first composition and a second composition that may be co-administered to a patient orally.

The co-administration of specific vitamin and mineral combinations and drugs may improve the prevention; treatment and/or alleviation of the occurrence or negative effects of cardiovascular disease. Co-administering to a patient of one unit dose of compositions comprising vitamin B6, vitamin B12, folic acid and CoQ10 and one unit dose of compositions comprising aspirin may occur at the same time; in either order 1 minute following the administration of the other; in either order 2 minutes following the administration of the other; in either order 3 minutes following the administration of the other; in either order 4 minutes following the administration of the other; in either order 5 minutes following the administration of the other; in either order 6 minutes following the administration of the other; in either order 7 minutes following the administration of the other; in either order 8 minutes following the administration of the other; in either order 9 minutes following the administration of the other; in either order 10 minutes following the administration of the other; in either order 11 minutes following the administration of the other; in either order 12 minutes following the administration of the other; in either order 13 minutes following the administration of the other; in either order 14 minutes following the administration of the other; in either order 15 minutes following the administration of the other; in either order 16 minutes following the administration of the other; in either order 17 minutes following the administration of the other; in either order 18 minutes following the administration of the other; in either order 19 minutes following the administration of the other; in either order 20 minutes following the administration of the other; in either order 21 minutes following the administration of the other; in either order 22 minutes following the administration of the other; in either order 23 minutes following the administration of the other; in either order 24 minutes following the administration of the other; in either order 25 minutes following the administration of the other; in either order 26 minutes following the administration of the other; in either order 27 minutes following the administration of the other; in either order 28 minutes following the administration of the other; in either order 29 minutes following the administration of the other; in either order 30 minutes following the administration of the other; in either order 31 minutes following the administration of the other; in either order 32 minutes following the administration of the other; in either order 33 minutes following the administration of the other; in either order 34 minutes following the administration of the other; in either order 35 minutes following the administration of the other; in either order 36 minutes following the administration of the other; in either order 37 minutes following the administration of the other; in either order 38 minutes following the administration of the other; in either order 39 minutes following the administration of the other; in either order 40 minutes following the administration of the other; in either order 41 minutes following the administration of the other; in either order 42 minutes following the administration of the other; in either order 43 minutes following the administration of the other; in either order 44 minutes following the administration of the other; in either order 45 minutes following the administration of the other; in either order 46 minutes following the administration of the other; in either order 47 minutes following the administration of the other; in either order 48 minutes following the administration of the other; in either order 49 minutes following the administration of the other; in either order 50 minutes following the administration of the other; in either order 51 minutes following the administration of the other; in either order 52 minutes following the administration of the other; in either order 53 minutes following the administration of the other; in either order 54 minutes following the administration of the other; in either order 55 minutes following the administration of the other; in either order 56 minutes following the administration of the other; in either order 57 minutes following the administration of the other; in either order 58 minutes following the administration of the other; in either order 59 minutes following the administration of the other; in either order 60 minutes following the administration of the other; in either order one administered about 2 hours following the administration of the other; in either order one administered about 3 hours following the administration of the other; in either order one administered about 4 hours following the administration of the other; in either order one administered about 5 hours following the administration of the other; in either order one administered about 6 hours following the administration of the other; in either order one administered about 7 hours following the administration of the other; in either order one administered about 8 hours following the administration of the other; in either order one administered about 9 hours following the administration of the other; in either order one administered about 10 hours following the administration of the other; in either order one administered about 11 hours following the administration of the other; in either order one administered about 12 hours following the administration of the other; in either order together once a day; in either order together twice a day; in either order together three times a day; in either order together four times a day; in either order together once every 2 days; in either order together once every 3 days; in either order together once every 4 days; in either order together once every 5 days; in either order together once every 6 days; or in either order together once a week.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A first composition of the following formulation was prepared in caplet form by standard methods known to those skilled in the art:

| | |
|---|---|
| Vitamin B₆ | 30 mg |
| Vitamin B₁₂ | 1.8 mg |
| Folic acid | 2.5 mg |
| CoQ₁₀ | 50 mg |

A second composition of the following formulation was prepared in caplet form by standard methods known to those skilled in the art:

| | |
|---|---|
| Aspirin | 81 mg |

### Example 2

A study is undertaken to evaluate the effectiveness of the compositions of the present invention in the treatment of patients. The objective of the study is to determine whether oral co-administration of the compositions results in an improvement of the nutritional status with regard to specific vitamins and minerals contained in the co-administered compositions.

A double-blind, placebo controlled study is conducted over a six-month period. A total of 120 subjects, aged 3045 years, are chosen for the study. An initial assessment of the nutritional status of each subject is conducted. Vitamin B₆ is measured by a radioenzymatic assay method wherein serum is incubated with apoenzyme tyrosine-decarboxylase, C₁₄ labeled tyrosine is added to start the enzymatic reaction which is stopped with HCl. Subsequently the free C₁₄-labelled CO₂ is adsorbed by a KOH impregnated filtering paper. The measured C₁₄ activity is directly proportional to the B₆ concentration. Vitamins B₁₂ and folic acid are measured by quantitative radioassay methods using purified intrinsic factor and purified folate binding protein. CoQ₁₀ is measured by using column-switching high performance liquid chromatography (HPLC). Determination of CoQ₁₀ is performed on a reversed-phase analytical column with ultraviolet detection at 275 nm. The mobile phase contains 10% (v/v) isopropanol in methanol at a flow rate of 1.5 m./min. This method allows for the detection of 0.1 microg/ml CoQ₁₀ in plasma (S/N=3).

The 120 subjects are separated into 4 separate groups of 30 subjects. In a first group comprising men, and in a second group comprising women, each subject is co-administered one dosage form of the first composition and one dosage form of the second composition as described in Example 1 once a day. In a third group comprising men and a fourth group comprising women, each subject is co-administered one placebo dosage form for each composition once a day. Thus, dosage form co-administration occurs every 24 hours. No other drugs or nutritional supplements are taken by the subjects during the assessment period.

An assessment of the nutritional status of each subject is conducted utilizing methods described above at one month intervals for a six month period. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 1, 2, 3, 4, 5 and 6 months, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status of all vitamin and mineral levels measured is observed in the treated subjects over the controls upon completion of the study. Therefore, the study confirms that oral co-administration of the two compositions of the present invention is effective in improving the nutritional status of patients.

While specific embodiments of the present invention have been described, other and further modifications and changes may be made without departing from the spirit of the invention. All further and other modifications and changes are included that come within the scope of the invention as set forth in the claims. The disclosure of all publications cited above are expressly incorporated by reference in their entireties to the same extent as if each were incorporated by reference individually.

What is claimed is:

1. A method comprising co-administering a first composition comprising vitamin B₆, vitamin B₁₂, folic acid, and CoQ₁₀ to a patient and a second composition comprising aspirin to said patient, in any order.

2. The method of claim 1, wherein said folic acid is selected from the group consisting of vitamin B₉, folacin, metafolin, folate, (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof.

3. The method of claim 1, wherein said first composition is substantially free of one or more of other added vitamins, added minerals and added coenzymes.

4. The method of claim 1, wherein said first composition further comprises a pharmaceutically acceptable carrier.

5. The method of claim 4, wherein said pharmaceutically acceptable carrier is selected from one or more of the group consisting of binders, diluents, lubricants, glidants, colorants, emulsifiers, disintegrants, starches, water, oils, alcohols, preservatives and sugars.

6. The method of claim 1, wherein said vitamin B₆ is present in the range of about 15 mg to about 45 mg.

7. The method of claim 1, wherein said vitamin B₁₂ is present in the range of about 0.9 mg to about 2.7 mg.

8. The method of claim 1, wherein said folic acid is present in the range of about 1.25 mg to about 3.75 mg.

9. The method of claim 1, wherein said CoQ₁₀ is present in the range of about 25 mg to about 75 mg.

10. The method of claim 1, wherein said first composition comprises about 15 mg to about 45 mg vitamin B₆; about 0.9 mg to about 2.7 mg vitamin B₁₂; about 1.25 mg to about 3.75 mg folic acid; and about 25 mg to about 75 mg CoQ₁₀.

11. The method of claim 1, wherein said first composition comprises about 24 mg to about 36 mg vitamin B₆; about 1.44 mg to about 2.16 mg vitamin B₁₂; about 2 mg to about 3 mg folic acid; and about 40 mg to about 60 mg CoQ₁₀.

12. The method of claim 1, wherein said first composition comprises about 27 mg to about 33 mg vitamin B₆; about 1.26 mg to about 1.98 mg vitamin B₁₂; about 2.25 mg to about 2.75 mg folic acid; and about 45 mg to about 65 mg CoQ₁₀.

13. The method of claim 12, wherein said vitamin B₆ is present in the amount of about 30 mg.

14. The method of claim 12, wherein said vitamin B₁₂ is present in the amount of about 1.8 mg.

15. The method of claim 12, wherein said folic acid is present in the amount of about 2.5 mg.

16. The method of claim 12, wherein said CoQ₁₀ is present in the amount of about 50 mg.

17. The method of claim 1, wherein said vitamin B₆ is present in the amount of about 30 mg; said vitamin B₁₂ is present in the amount of about 1.8 mg; said folic acid is present in the amount of about 2.5 mg; and said CoQ₁₀ is present in the amount of about 50 mg.

18. The method of claim 1, wherein said first composition is administered to said patient orally.

19. The method of claim 1, wherein said first composition is administered to said patient to prevent, treat and/or alleviate the occurrence or negative effects of cardiovascular disease.

20. The method of claim 19, wherein said cardiovascular disease is selected from one or more of the group consisting of congestive heart failure, ischemic heart disease, cardiomyopathy, and hypertension.

21. The method of claim 1, wherein said second composition is substantially free of one or more of other added vitamins, added minerals and added coenzymes.

22. The method of claim 1, wherein said second composition further comprises a pharmaceutically acceptable carrier.

23. The method of claim 22, wherein said pharmaceutically acceptable carrier is selected from one or more of the group consisting of binders, diluents, lubricants, glidants, colorants, emulsifiers, disintegrants, starches, water, oils, alcohols, preservatives and sugars.

24. The method of claim 1, wherein said aspirin is present in the amount of about 40.5 mg to about 121.5 mg.

25. The method of claim 1, wherein said aspirin is present in the amount of about 64.8 mg to about 97.2 mg.

26. The method of claim 1, wherein said aspirin is present in the amount of about 72.9 mg to about 89.1 mg.

27. The method of claim 1, wherein said aspirin is present in the amount of about 81 mg.

28. The method of claim 1, wherein said second composition is administered to said patient orally.

29. The method of claim 1, wherein said second composition is administered to said patient to prevent, treat and/or alleviate the occurrence or negative effects of cardiovascular disease.

30. The method of claim 29, wherein said cardiovascular disease is selected from one or more of the group consisting of ischemic heart disease, coronary thrombosis, angina, myocardial infarction, and stroke.

31. The method of claim 1, further comprising co-administering a cholesterol-lowering drug.

32. The method of claim 31, wherein said cholesterol-lowering drug is selected from the group consisting of Crestor®, Lipitor®, Lescol®, Mevacor® and Zocor®.

33. The method of claim 31, further comprising co-administering said cholesterol-lowering drug with said first composition.

34. The method of claim 31, further comprising co-administering said cholesterol-lowering drug with said second composition.

35. The method of claim 31, further comprising co-administering said cholesterol-lowering drug with said first and said second composition.

36. A kit comprising:
a first composition comprising vitamin B₆, vitamin B₁₂, folic acid, and CoQ₁₀; and
a second composition comprising aspirin;
wherein said first and second compositions are packaged for co-administration to a patient, in any order.

37. The kit of claim 36, wherein said folic acid is selected from the group consisting of vitamin B₉, folacin, metafolin, folate, (6S)-tetrahydrofolic acid or a polyglutamyl derivative thereof, 5-methyl-(6S)-tetrahydrofolic acid or a polyglutamyl derivative thereof, 5-formyl-(6S)-tetrahydrofolic acid or a polyglutamyl derivative thereof, 10-formyl-(6R)-tetrahydrofolic acid or a polyglutamyl derivative thereof, 5,10-methylene-(6R)-tetrahydrofolic acid or a polyglutamyl derivative thereof, 5,10-methenyl-(6R)-tetrahydrofolic acid or a polyglutamyl derivative thereof and 5-formimino-(6S)-tetrahydrofolic acid or a polyglutamyl derivative thereof.

38. The kit of claim 36, wherein said first composition is substantially free of one or more of other added vitamins, added minerals and added coenzymes.

39. The kit of claim 36, wherein said first composition further comprises a pharmaceutically acceptable carrier.

40. The kit of claim 39, wherein said pharmaceutically acceptable carrier is selected from one or more of the group consisting of binders, diluents, lubricants, glidants, colorants, emulsifiers, disintegrants, starches, water, oils, alcohols, preservatives and sugars.

41. The kit of claim 36, wherein said vitamin B₆ is present in the range of about 15 mg to about 45 mg.

42. The kit of claim 36, wherein said vitamin B₁₂ is present in the range of about 0.9 mg to about 2.7 mg.

43. The kit of claim 36, wherein said folic acid is present in the range of about 1.25 mg to about 3.75 mg.

44. The kit of claim 36, wherein said CoQ₁₀ is present in the range of about 25 mg to about 75 mg.

45. The kit of claim 36, wherein said first composition comprises about 15 mg to about 45 mg vitamin B₆; about 0.9 mg to about 2.7 mg vitamin B₁₂; about 1.25 mg to about 3.75 mg folic acid; and about 25 mg to about 75 mg CoQ₁₀.

46. The kit of claim 36, wherein said first composition comprises about 24 mg to about 36 mg vitamin B₆; about 1.44 mg to about 2.16 mg vitamin B₁₂; about 2 mg to about 3 mg folic acid; and about 40 mg to about 60 mg CoQ₁₀.

47. The kit of claim 36, wherein said first composition comprises about 27 mg to about 33 mg vitamin B₆; about 1.26 mg to about 1.98 mg vitamin B₁₂; about 2.25 mg to about 2.75 mg folic acid; and about 45 mg to about 55 mg CoQ₁₀.

48. The kit of claim 47, wherein said vitamin B₆ is present in the amount of about 30 mg.

49. The kit of claim 47, wherein said vitamin B₁₂ is present in the amount of about 1.8 mg.

50. The kit of claim 47, wherein said folic acid is present in the amount of about 2.5 mg.

51. The kit of claim 47, wherein said CoQ₁₀ is present in the amount of about 50 mg.

52. The kit of claim 36, wherein said vitamin B₆ is present in the amount of about 30 mg; said vitamin B₁₂ is present in the amount of about 1.8 mg; said folic acid is present in the amount of about 2.5 mg; and said CoQ₁₀ is present in the amount of about 50 mg.

53. The kit of claim 36, wherein said first composition is administered to said patient orally.

54. The kit of claim 36, wherein said first composition is administered to said patient to prevent, treat and/or alleviate the occurrence or negative effects of cardiovascular disease.

55. The kit of claim 54, wherein said cardiovascular disease is selected from one or more of the group consisting of congestive heart failure, ischemic heart disease, cardiomyopathy, and hypertension.

56. The kit of claim 36, wherein said second composition is substantially free of one or more of other added vitamins, added minerals and added coenzymes.

57. The kit of claim 36, wherein said second composition further comprises a pharmaceutically acceptable carrier.

58. The kit of claim 57, wherein said pharmaceutically acceptable carrier is selected from one or more of the group consisting of binders, diluents, lubricants, glidants, colorants, emulsifiers, disintegrants, starches, water, oils, alcohols, preservatives and sugars.

59. The kit of claim 36, wherein said aspirin is present in the amount of about 40.5 mg to about 121.5 mg.

60. The kit of claim 36, wherein said aspirin is present in the amount of about 64.8 mg to about 97.2 mg.

61. The kit of claim 36, wherein said aspirin is present in the amount of about 72.9 mg to about 89.1 mg.

62. The kit of claim 36, wherein said aspirin is present in the amount of about 81 mg.

63. The kit of claim 36, wherein said second composition is administered to said patient orally.

64. The kit of claim 36, wherein said second composition is administered to said patient to prevent, treat and/or alleviate the occurrence or negative effects of cardiovascular disease.

65. The kit of claim 64, wherein said cardiovascular disease is selected from one or more of the group consisting of ischemic heart disease, coronary thrombosis, angina, myocardial infarction, and stroke.

66. The kit of claim 36, wherein said packaging is selected from the group consisting of bottles and blister packs.

67. The kit of claim 66, wherein said bottles are sold together; one bottle containing said first composition and a second bottle containing said second composition.

68. The kit of claim 66, wherein said bottles are sold separately; one bottle containing said first composition and a second bottle containing said second composition.

69. The kit of claim 66, wherein said bottles containing said first composition and said second composition are advertised as more effective if co-administered.

70. The kit of claim 69, wherein said advertisements are selected from one or more of the group consisting of internet, print, and product packaging advertisements.

71. The kit of claim 66, wherein said blister packs are sold together and said blister packs comprise a first blister pack containing said first composition and a second blister pack containing said second composition.

72. The kit of claim 66, wherein said blister pack contains both said first composition and said second composition paired together per unit dose.

73. The kit of claim 66, wherein said blister packs are sold separately and said blister packs comprise a first blister pack containing said first composition and a second blister pack containing said second composition.

74. The kit of claim 66 wherein said blister packs containing said first composition and said second composition are advertised as more effective if co-administered.

75. The kit of claim 74 wherein said advertisements are selected from one or more of the group consisting of internet, print, and product packaging advertisements.

76. The kit of claim 36, wherein said first composition and said second composition are co-administered to a patient.

77. The kit of claim 76, wherein said first composition and said second composition are co-administered to said patient orally.

78. The kit of claim 36, wherein said first composition said second composition are co-administered to a patient to prevent, treat and/or alleviate the occurrence or negative effects of cardiovascular disease.

79. The kit of claim 78, wherein said cardiovascular disease is selected from one or more of the group consisting of congestive heart failure, ischemic heart disease, cardiomyopathy, hypertension, coronary artery disease, high blood pressure, arrhythmia, thrombogenicity, angina, myocardial infarction, and stroke.

80. The kit of claim 36, further comprising co-administering a cholesterol-lowering drug.

81. The kit of claim 80, wherein said cholesterol-lowering drug is selected from the group consisting of Crestor®, Lipitor®, Lescol®, Mevacor® and Zocor®.

82. The kit of claim 80, further comprising co-administering said cholesterol-lowering drug with said first composition.

83. The kit of claim 80, further comprising co-administering said cholesterol-lowering drug with said second composition.

84. The kit of claim 80, further comprising co-administering said cholesterol-lowering drug with said first and said second composition.

85. A method comprising providing the kit of claim 36 to patients.
